(19) United States
(12) Patent Application Publication
(10) Pub. No.: US 2004/0260196 A1
(43) Pub. Date: Dec. 23, 2004

(54) Method and apparatus for measuring local body fat by using bioelectrical impedance technology

(76) Inventor: Chih-Cheng Lu, Kaohsiung (TW)

Correspondence Address: Rabin & Berdo, P.C., Suite 500, 1101 14th Street, Washington, DC, 20005, US

(21) Appl. No.: 10/463,331

(22) Filed: Jun. 18, 2003

(51) Int. Cl. A61B 5/05

(52) U.S. Cl. 600/547

(57) ABSTRACT

A method and apparatus for measuring local body fat using the bioelectrical impedance analysis where a flat bottom plane of the impedance-measuring apparatus has four electrodes to connect to a pre-selected segment of the body to measure the body fat content, which allows all four electrodes on the bottom plane to be connected to the human body at the same time. When the impedance-measuring apparatus is connected to the person tested, a weak current is sent through the current electrodes and the body tissue. Since the resistance characteristics of body fat mass and fat free mass is quite different, the resistance values measured from segments of the body can provide useful information for computation of the proportion of fat free mass and body fat mass in the body.

### BACKGROUND OF THE INVENTION

1. Field of the Invention

The present invention relates to a method and apparatus for measuring local body fat by using the bioelectrical impedance analysis. The impedance-measuring apparatus has four electrodes on a flat bottom plane for contacting pre-selected segment of the human body to check for the body fat content.

2. Description of Related Art

A human body is mainly composed of water, protein, bone and fat. In medical terms, the fat is called body fat mass, and muscle is called fat free mass, and is the primary means of supporting and controlling the human body. There are a variety of body fat measuring techniques in use. Conventional techniques include measuring with a caliper, Infra-red Detection, Hydrodensimeter, Nuclear Magnetic Resonance (NMR), and Dual Energy X-ray Absorptiometer (DEXA). Some of these are quite expensive to carry out, and trained technicians are needed to measure the thickness of the sub-cutaneous fatty tissue layer with a caliper. Due to variations in skin texture and the distribution of fatty tissues in different people, the accuracy of determining body fat is directly related to the skillfulness of the person using the instrument. The infra-red detection technique based on optical measurement could be biased by factors such as skin color and skin conditions of the person being tested, and cannot be performed in sunlight or bright illumination. Further, such infra-red instruments need to be frequently recalibrated to maintain their accuracy.

Bioelectrical impedance analysis is a widely used technique for measuring body fat content. Bioelectrical impedance analysis is based on the different resistance characteristics exhibited by different components of the body. For instance, the resistance to electrical current through the body fat mass is higher than that through the fat free mass.

The aim of the present invention is to overcome the disadvantages associated with conventional body fat measuring techniques.

### SUMMARY OF THE INVENTION

The main objective of the present invention is to provide a method and apparatus for measuring local body fat using bioelectrical impedance analysis. The impedance-measuring apparatus has four electrodes on a flat bottom plane for contacting pre-selected segment of the body to check for the body fat content, such that all four electrodes on the bottom plane can be connected to a segment of the body at the same time. Two of the electrodes are current electrodes, and two are voltage electrodes.

When to the impedance-measuring apparatus is connected a person, a weak alternating current flows through the body tissue from one of the current electrodes and then back to the other current electrode. The voltage across the body tissue is measured by the voltage electrodes. The proportion of fat in the body tissue can thus be computed as a function of the measured voltage.

Another objective of the present invention is to provide a method and apparatus for measuring local body fat using the bioelectrical impedance analysis, whereby the resistance values gathered from several segments of the body can be used to estimate fat content of the whole body.

Other advantages and features of the invention will become apparent from the detailed description when taken in conjunction with the attached drawings.

### BRIEF DESCRIPTION OF THE DRAWINGS

FIG. 1 is a perspective view of a body fat measuring apparatus in accordance with the present invention;

FIG. 2 is a bottom view of the body fat measuring apparatus in FIG. 1;

FIG. 3 is a perspective view of a person in a standing position to have body fat in the upper part of the body checked; and

FIG. 4 is an operational bottom plan view of the body fat measuring apparatus in FIG. 1 showing the correct way of placing fingers over the electrodes for checking body fat in the upper part of the body.

### DETAILED DESCRIPTION OF THE PREFERRED EMBODIMENT

The present invention provides a method and apparatus for measuring local body fat in the human body with bioelectrical impedance analysis. Since resistance to electrical current through body fat mass is higher than that through fat free mass, a small current can be sent through a segment of the body to measure the body fat content, and the voltage across the current electrodes is then measured. The proportion of body fat mass and fat free mass can thus be computed as a function of the measured voltage value.

The bioelectrical impedance in the body tissue can be expressed by the basic formula:

Z=ρ×(L/A) (1)

where Z: tissue impedance

ρ: resistivity of the body tissue;

L: distance between voltage measuring electrodes; and

A: cross-sectional area of the segment being checked.

If the human body is considered as a cylindrical model, and the height of the body is represented by L, then the weight of the body can be expressed as a product of the cross-sectional area (A) of the body multiplied by height (L) and tissue density (d). Therefore, the above expression can be rewritten as

Z=ρ(h²×d)/(A×h×d) (2)

where,

Z: resistance of the body tissue being checked;

ρ: resistivity of the body tissue being checked;

h: height of the local object (body tissue) being checked;

A: cross-sectional area of the local object (body tissue) being checked; and

d: tissue density, which is determined from the ration of total body weight to height.

The proportion of fat free mass (W) in the cylindrical model can be expressed as

W=A×h×d (3)

thus, the Z expression can be rewritten as

Z=ρ(h²×d/W) (4)

W=ρ×d(h²/Z) (5)

Therefore, the proportion of fat free mass can be computed from the height, tissue impedance and density. Formulae (4) and (5) form the basis for computation of the body fat content in accordance with the present invention.

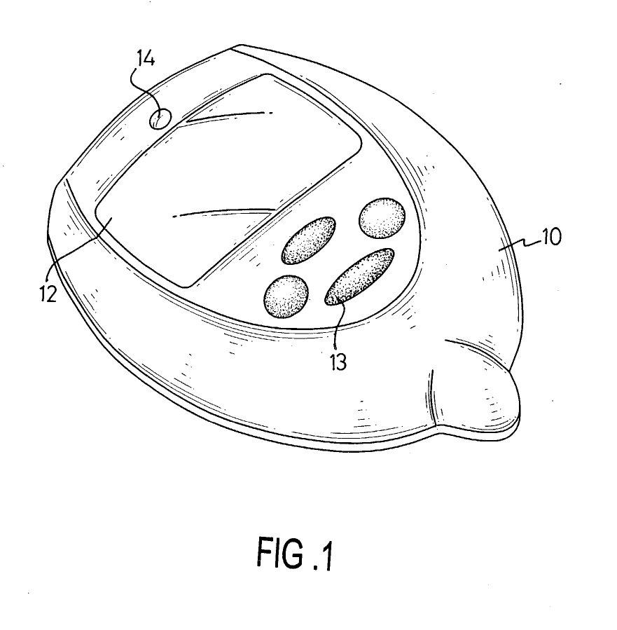

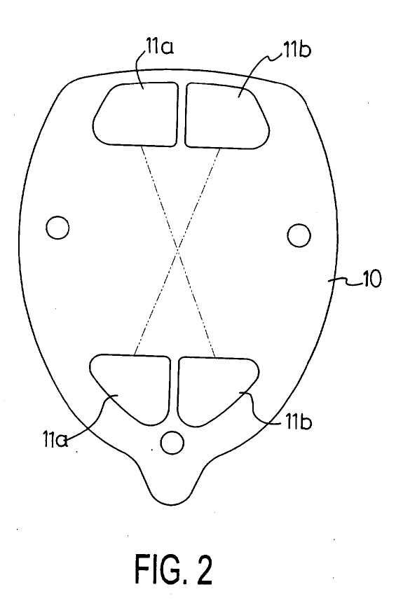

With reference to FIG. 1 and 2, the impedance-measuring instrument comprises a main body (10) with a front panel (not numbered), a display (12), function keys (13), an operation indicator (14), a bottom plane (not numbered) and four electrodes (11a, 11b). The electrodes (11a, 11b) are mounted on the bottom plane and are separated into a first electrode pair and a second electrode pair. These two electrode pairs each are paired up by a current electrode (11a) and a voltage electrode (11b) in alternate positions. In other words, the first pair has the current electrode (11a) on the left and the voltage electrode (11b) on the right, while the second pair has the voltage electrode (11b) on the left and the current electrode (11a) on the right. Therefore, electrodes are lined up on diagonal lines (as depicted by dotted line in the diagram) of a quadrangle where these four electrodes (11a, 11b) occupying all corners. The readout display (12) is mounted on the front panel of the main body (10), together with a number of function keys (13) for selection of the operation modes, and the operation indicator (14).

When measuring the impedance, four electrodes (11a, 11b) are placed squarely over the segment of the body to be checked for local body fat, such that all four electrodes on the bottom plane are in contact with the pre-selected segment in the same plane. A small current is sent from one of the current electrode (11a) into and through the body tissue, to the other current electrode and back to the measuring apparatus. The voltage across the tissue is measured by the voltage electrode (11b). Since the resistance to electrical current by a fat free mass and a body fat mass are different, the fat content in the body tissue can be computed as a function of the measured voltage value. Besides the basic capability of measuring local body fat, the impedance-measuring apparatus can also be used for measuring fat content in the upper part of the body or the whole body. The impedance measuring apparatus includes the following functions.

(1) Measuring local body fat: A function key (13) on the front panel of the impedance-measuring apparatus (10) is first pressed to select the local checking mode, and then all four electrodes (11a, 11b) are placed squarely over a segment of the body such as the arm, abdomen, leg, etc.. When the impedance-measuring apparatus (10) is connected to the person, an electrical current passes through the pre-selected part of the body tissue, and the resistance to electrical current through the body tissue is measured for further computation of fat content.

(2) Measuring body fat in the whole body: After two or more resistance values from different segments of the body (for example, arm, abdomen, and leg) are collected, the resistance values are then averaged out to derive an estimated body content for the whole body.

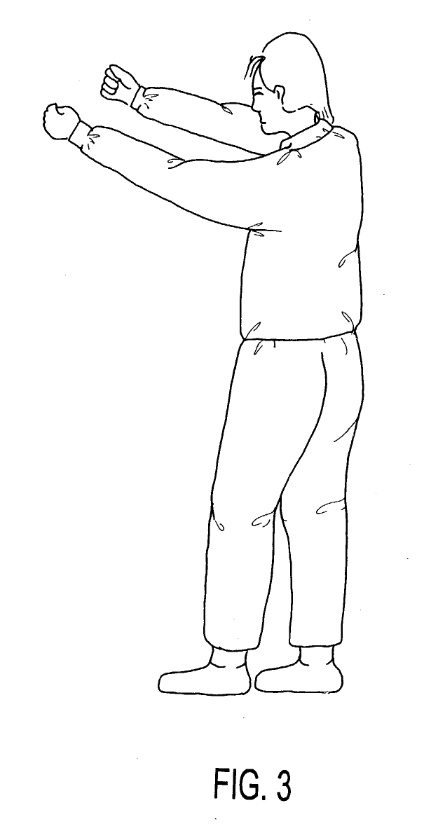

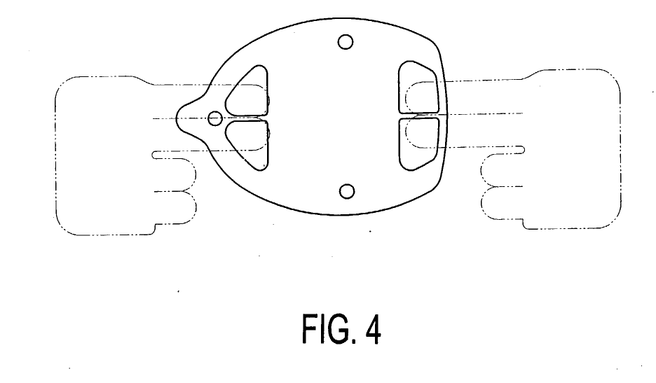

(3) Measuring body fat in the upper part of the body: With reference to FIG. 3, the person being tested has to be in a standing position with both arms stretched out horizontally in front of the body. The index finger and middle finger on both hands are placed over the four electrodes (11a, 11b) to measure the resistance through the upper part of the body. The current is sent through one arm of the body and through the upper part of the body and back to the impedance-measuring instrument (10) through the other arm, thus forming a closed circuit. The voltage across the current electrodes (11a) is measured for computation of fat content in the upper part of the body. With reference to FIG. 4A, the index finger and the middle finger in contact with the electrodes (11a, 11b) during impedance-measuring must be separated.

Some of the design features of the impedance measuring apparatus are highlighted below.

(1) The distance between the first electrode pair and the second electrode pair has been optimized in accordance with the present invention. The electrode distance is one of the crucial factors affecting the precision of impedance measurement, since it is related to the size of test objects suitable for the measuring apparatus.

(2) The weight of the impedance measuring apparatus has been optimized to allow for placement squarely over any body segments to be checked for body fat, such that all four electrodes on the bottom plane can be connected to the pre-selected body segment at the same time.

(3) Since the diagonal crossing angle of the current electrode pair and the voltage electrode pair is crucial to the precision of impedance measurement, the crossing angle has been optimized.

(4) The crosswise configuration of the current electrodes and voltage electrodes in accordance with the present invention makes it possible to put together a measuring apparatus small enough for clipping over any segment of the body to check for the fat content.

The measuring method in accordance with the present invention can be concluded to as comprises the following steps.

1. Placing the impedance measuring instrument squarely over the body region to be checked.

2. Sending a small current through the body tissue of the pre-selected segment.

3. Measuring the voltage across the tissue of the pre-selected segment.

4. Computing the fat content of the body tissue as a function of the measured voltage.

The impedance-measuring apparatus is capable of checking local body fat, as well as the fat content in the upper part of the body or even the whole body. It is a versatile impedance-measuring instrument and suitable for people watching their weight or trying to keep physically fit.

The foregoing illustration of the preferred embodiments of the present invention is intended to be illustrative only. Under no circumstances should the scope of the present invention be restricted the embodiment illustrated.

What is claimed is:

1. A method for measuring local body fat using the bioelectrical impedance analysis, comprising the steps of:
placing tetrapolar electrodes over a body tissue to be measured;
sending a small current through the body tissue;
measuring the voltage across the body tissue; and
computing the fat content of the local body tissue as a function of the measured voltage, wherein by changing the position of the body tissue to be measured, more than one fat content reading of an entire body are obtained.

2. The method as claimed in claim 1, wherein the effective measuring area of the local body tissue is in a range from 1.5 cm diameter to 10 cm diameter.

3. The method as claimed in claim 1, wherein the tetrapolar electrodes comprises two current electrodes and two voltage electrodes.

4. The method as claimed in claim 3, wherein the two current electrodes and two voltage electrodes are disposed crosswise, such that the first electrode pair is composed of a current electrode and a voltage electrode and the second electrode pair likewise but in alternate positions.

5. The method as claimed in claim 1, wherein for computation of global impedance at least two resistance values have to be taken from different segments of the body in order to estimate the fat content in the upper part of the body or the whole body.

6. An apparatus for measuring local body fat using the bioelectrical impedance analysis, comprising a main body which is the impedance-measuring instrument, multiple current electrodes and voltage electrodes fixed on the bottom plane, and a readout display and multiple function keys on the front panel of the main body.

7. The apparatus as claimed in claim 6, wherein the first electrode pair and the second electrode pair are fixed on opposite ends of the bottom plane, such that each has a current electrode and a voltage electrode paired up, and the two voltage electrodes and the two current electrodes are in crosswise arrangement, making like kind electrodes lined up on the diagonal lines of a quadrangle.
